(19) United States
(12) Patent Application Publication
(10) Pub. No.: US 2024/0382395 A1
(43) Pub. Date: Nov. 21, 2024

(54) DENTAL CARE PRODUCT

(71) Applicant: DR. KURT WOLFF GMBH & CO. KG, Bielefeld (DE)

(72) Inventors: Frederic MEYER, Bielefeld (DE); Joachim ENAX, Bielefeld (DE); Erik SCHULZE ZUR WIESCHE, Bielefeld (DE)

(73) Assignee: DR. KURT WOLFF GMBH & CO. KG, Bielefeld (DE)

(21) Appl. No.: 18/272,382

(22) PCT Filed: Jan. 11, 2022
(86) PCT No.: PCT/DE2022/100007

§ 371 Date: Jul. 14, 2023

(30) Foreign Application Priority Data
Jan. 15, 2021 (DE) 10 2021 100 811.5

(51) Int. Cl. A61K 8/24; A61Q 11/00

(52) U.S. Cl. CPC A61K 8/24; A61Q 11/00; A61K 2800/30; A61K 2800/34

(57) ABSTRACT

A dental care product, in particular a toothpaste, containing calcium phosphate compounds is free of fluorine compounds and free of silicon compounds and contains at least one surfactant selected from taurates, glycinates or sarcosinates. The dental care product remineralizes smaller caries lesions and/or repairs microfine defects in the tooth enamel, in particular also in patients with dry mouth/saliva deficiency.

The present invention relates to a dental care product, in particular a toothpaste, containing at least one calcium phosphate compound.

The term “dental care product” within the meaning of the present patent application is understood to mean toothpastes, dental gels and polishing pastes.

A toothpaste, also referred to as dental cream, may be used in combination with a toothbrush for mechanical tooth cleaning and is a soft or semi-solid composition for oral application, particularly to the teeth and/or gums.

Dental care is becoming more and more important if only because of the increasingly carbohydrate-rich diet worldwide. Thus, in addition to aesthetic aspects, special emphasis is increasingly being placed on preventive care, wherein the focus here is primarily on reducing or even avoiding plaque, caries and/or halitosis (bad breath), as well as healthy gums.

One of the characteristics of the gums is that they surround the teeth cervically. The gums surround the neck of the tooth, which seals the entry point of the tooth into the jawbone of the oral cavity. The gum thus serves, among other things, to protect and hold the tooth in place.

The different parts of a natural tooth are the crown, neck and root, wherein these are made up of several layers. Of these layers, normally only the outer enamel (enamelum) is visible, which encloses the dentin and other layers. To be able, for example, to bite or grind food without damaging the teeth, the enamel is very hard. It consists of about 97% by weight of hydroxylapatite (HAP), which has the empirical formula Ca₅(PO₄)₃(OH). Dentin is also considered to be the hard substance of the tooth and consists of about two thirds likewise of hydroxylapatite. In addition to hydroxylapatite, however, dentin also contains proteins (for example collagen) and water and is therefore softer than the enamel.

Dental diseases, such as caries, can be based on the formation of bacterial microfilms and/or bacterial inflammation. Although often avoidable by taking precautions, caries remains one of the most common chronic diseases in children and there is a great need for products that prevent and cure tooth decay in poorer children worldwide.

It has been documented that saliva has a remineralizing effect due to its supersaturation of Ca²⁺ and PO₄³⁻ ions in bioavailable form. However, the remineralizing effect caused by saliva is not only slow, but also apparently insufficient to protect individuals from caries and/or to remineralize already existing dental lesions without the addition of remineralization-enhancing additives.

Caries is considered to be a disease dependent on several influences, in particular of tooth enamel and dentin, with the involvement of microorganisms. Thus, in a first step, a deposit, also called a pellicle, can form on the tooth surface from saliva protein, among other things. The rougher the tooth surface, the easier it is for the deposit to form on or adhere to it. After some time, this pellicle forms a film that covers the tooth surface and is a few micrometers thick. Bacteria can then continue to multiply and spread on this film, wherein this film can be regarded as a biofilm. Furthermore, the bacteria mentioned can produce organic acids, such as gluconic and lactic acid, from low-molecular carbohydrates often found in today's food. Both these organic acids produced by bacteria and the acids supplied via food, such as fruit acids, can cause erosion of the hard tooth structure. Among other things, calcium phosphates are dissolved from the enamel. If this process is not stopped or, preferably, even reversed, it can lead to demineralization of the enamel and possibly also of the dentin after some time. A defect in the hard tooth structure that occurs in this way is called a carious lesion, wherein, for example, carious lesions on the dentin layer can be recognized by a yellow to brown coloration of the corresponding part of the tooth. Thus, while carious lesions on the enamel alone can often still be reduced or eliminated by re-mineralizing the enamel, in the case of carious lesions that affect other layers besides the enamel, the damaged part of the tooth is usually removed and the resulting hole (caries lesion) is closed using a filling material. In summary, it can be said that caries formation can be promoted by the interaction of the following factors:
specific bacteria adhering to the pellicle; low tooth quality or mineral quality of the tooth plaque
food containing carbohydrates of low molecular weight
time: research also suggests that the hydroxylapatite nanoparticles produced by the natural abrasion of enamel can interfere with biofilm management, wherein this abrasion has greatly decreased with the switch from protein-rich to carbohydrate-rich diets.

Accordingly, one possibility for caries prophylaxis could be the supply of calcium phosphate compounds, in particular hydroxylapatite. In addition, there are various approaches that all aim to prevent caries in order to avoid the aforementioned treatments. Caries prophylaxis with fluoride-containing dental care products can be considered to be the current standard. J. M. ten Cate: “Contemporary perspective on the use of fluoride products in caries prevention”, British Dental Journal, 23 Feb. 2013, vol. 214, no. 4, pp. 161-167, describes in a review the use and mode of action of fluoride-containing dental care products, such as toothpaste and mouthwash. In particular, it is pointed out that the reduction of caries with these fluoride-containing dental care products is achieved by their regular use. As a model, it is assumed here that fluoride accelerates the natural remineralization from saliva.

Various fluoride compounds, such as sodium fluoride, stannous fluoride, amine fluorides and monofmorphosphates, are suitable fluoride sources in the dental care products mentioned. J. M. ten Cate: “The Need for Antibacterial Approaches to Improve Caries Control”, Adv Dent Res 21:8-12, August 2009, p. 8-12, deals with the fact that fluoride provision alone may not be sufficient to achieve sufficient caries prevention, since, as mentioned above, bacteria also play a decisive role in caries formation. However, the fluoride ion itself does not demonstrate a pronounced antimicrobial effect against bacteria that can cause caries. For this reason, compositions for caries prophylaxis are being considered that, in addition to fluoride, also contain one or more antimicrobial substance(s). An example of this is chlorhexidine (CHX), the antimicrobial effect of which in the oral cavity has been investigated in many studies.

However, the use of fluorides in dental care products is also controversially discussed, since negative side effects are feared. One of these is so-called dental fluorosis, which is caused by excessive fluoride intake during tooth formation. Current toxic effects include nausea, vomiting and diarrhoea. Other examples are bone fluorosis, which is manifested by thickening of the outer bone layer and the associated loss of elasticity and resilience of the bones, and enamel fluorosis, which is recognizable by the appearance of whitish enamel spots on the tooth surface. In addition, it has been reported that the swallowing of high-dose dental care products can trigger acute fluoride poisoning, especially in children, which can occasionally even be fatal (Bashash, M. et al. Prenatal fluoride exposure and cognitive outcomes in children at 4and 6-12 years of age in Mexico. Environ. Health Perspect. 125, 097017 (2017)). Furthermore, it is reported that the WHO is unable to set a value for a daily fluoride requirement because fluoride is not an essential trace element and thus there are no diagnostic parameters and no evidence for the existence of clinical symptoms of “fluoride deficiency”.

The use of an antimicrobial substance, such as chlorhexidine, is also controversial in dental care, in particular caries prophylaxis. It has been reported that a relevant effect for caries prophylaxis is not always reproducible and only occurs in a portion of the cases studied. Furthermore, the antimicrobial effect of chlorhexidine is not limited to the oral bacteria involved in caries formation, but also includes beneficial bacteria. Furthermore, long-term treatment with chlorhexidine-containing products leads to undesirable side effects, such as tooth discoloration and taste disorders. Biomimetic tooth and mouth rinses with artificial enamel can contain, for example, zinc carbonate hydroxylapatite. This zinc carbonate hydroxylapatite is also commercially known as microrepair. Zinc carbonate hydroxylapatite-based products can reduce the initial bacterial colonization on the enamel surface without having antimicrobial properties that may disturb the ecological balance of the oral cavity. Moreover, these products are said, among other things, to remineralize and repair microfine defects in the enamel and form a protective layer.

Due to the above limitations of both the salivary homeostatic mechanism and fluoride addition-based approaches for caries prevention and remineralization, there is a need for alternative strategies that are at least equivalent to the efficiency of fluoride addition in remineralization, but without the corresponding undesirable side effects.

Furthermore, there are biometric dental care products based on the use of hydroxylapatite. For example, DE 10 2020 001 823 A1, WO 2018/024649 A1, DE 10 2017 009 626 A1 and DE 10 2018 102 365 describe oral care compositions containing synthetic hydroxylapatite, wherein hydroxylapatite, as indicated above, is a bioactive and biocompatible material with a similar chemical composition to the apatite of human tooth enamel. However, these compositions contain cetylpyridinium chloride, which is disadvantageous because of its bitter taste.

However, there is still a need for the use of a dental care product for the treatment or prevention of various diseases affecting the teeth, wherein this product can also be used for remineralization of lesions lying below the surface region. In other words, the use of the dental care product is intended to provide not only a means of prevention or treatment of numerous dental diseases, in particular caries, but also remineralization in deeper layers or cavities of the tooth enamel (deep mineralization), which cannot be provided by conventional products, in particular those containing fluoride.

Toothpaste according to the prior art contains abrasives, also referred to as cleaning bodies or abradants, which usually remove plaque and harmful bacteria from the tooth surface together with the toothbrush during the tooth cleaning process and can also provide a lightening (whitening) effect. Abrasive substances or cleaning bodies are contained in toothpastes in a quantity of up to 15% by weight or more, in relation to the total weight of the toothpaste. Examples of abrasives are whiting, marble powder and/or silicate compounds such as silica. The most commonly used worldwide are poly silicas (hydrated silica/silica) and calcium carbonate. The disadvantage is that, with the exception of calcium phosphate compounds (for example hydroxylapatite), all types of cleaning bodies are foreign to the body and do not correspond to the natural tooth mineral (hydroxylapatite). For example, cleaning bodies with a high relative hardness can irreparably damage the hard tooth structure. If the cleaning body has too low a hardness, the cleaning power of the toothpaste total formulation may be too low to effectively remove plaque during tooth brushing (increased risk of caries and periodontitis).

Furthermore, the use of the product should not significantly disturb the ecological balance in the oral region and/or risk discoloration of the teeth or disturbance of taste.

The object of the invention is to provide a dental care product, in particular a toothpaste, comprising one or more biomimetic or bioinspired active ingredient(s), in particular for the prophylaxis of caries, which avoids the disadvantages of the prior art described above. In particular, a dental care product, in particular a toothpaste, is to be provided which remineralizes already existing, smaller caries lesions and/or repairs microfine defects in the tooth enamel. Furthermore, a protective layer is to be applied to the tooth and/or open dentinal tubules are to be sealed. For this purpose, the above-mentioned effects are to be achieved, wherein the disadvantages that may be associated with the use of fluoride are to be avoided. Furthermore, the adhesion of bacteria to the enamel is to be advantageously reduced without significantly disturbing the ecological balance in the oral cavity and/or risking tooth discoloration or taste disturbance. The dental care product should also have no irritating effect on the mucous membranes.

These objects are achieved in a dental care product, in particular a toothpaste, according to the preamble in that the dental care product is free from fluoride compounds and free from silicon compounds and contains at least one surfactant selected from the group consisting of taurates, glycinates and sarcosinates.

The dental care product according to the invention is preferably in the form of a toothpaste. It contains no fluoride compounds and is thus fluoride-free. The above-described problems of fluoride-containing toothpastes (dental fluorosis, bone fluorosis, impairment of neuronal development of children in the womb (Bashash, M. et al. Prenatal fluoride exposure and cognitive outcomes in children at 4 and 6-12 years of age in Mexico. Environ. Health Perspect. 125, 097017 (2017)), etc.) are thus avoided.

The dental care product according to the invention is also free from silicon compounds (for example silica/polysilicic acid/hydrated silica). Surprisingly, it has been found in the context of the invention that the calcium phosphate compounds alone perform an excellent cleaning function, so that the presence of silicon compounds in the dental care product can be dispensed with entirely. With such a dental care product, excellent caries protection is achieved without mechanically damaging the teeth, as happens, for example, with conventional cleaning bodies in the form of polysilicic acids, aluminum oxide, perlite, titanium dioxide, etc.

Furthermore, the dental care product according to the invention contains at least one surfactant selected from the group consisting of taurates, glycinates and sarcosinates. Suitable surfactants may be anionic, non-ionic, cationic and zwitterionic surfactants, wherein non-ionic surfactants are often avoided because of their adverse effects on the antibacterial compound; likewise, cationic and zwitterionic surfactants are often avoided because they stain or darken the teeth.

The dental care product according to the invention is free from sodium lauryl sulphate and cetylpyridinium chloride and instead contains other surfactants that do not irritate the mucous membranes, such as taurates, preferably sodium methyl cocoyl taurate, glycinates, preferably sodium cocoyl glycinate, and sarcosinates, preferably sodium lauroyl sarcosinate. The specification of the various surfactants also includes all variants thereof (different alkyl chains, branches in the alkyl chains, counterions, etc.). Surfactants are important for a toothpaste because they help to distribute the active ingredients in the oral cavity during tooth brushing. Surfactants are also important for consumer convenience.

Sodium lauryl sulphate-free and cetylpyridinium chloride-free dental care products may be important, for example, for people with sensitive or irritated gums (for example children, gingivitis/periodontitis patients, people with xerostomia/hyposalivation). To combat plaque, sodium lauryl sulphate and silica are the most potent active ingredients in cosmetic oral care preparations. In the present invention, the disadvantages of these two active ingredients (sodium lauryl sulphate: irritating to the mucous membranes; silica: potentially damaging to teeth, not remineralizing) have been able to be overcome for the first time; i.e. cleaning that is equally gentle for the mucous membranes and tooth, but effective, with simultaneous effective remineralizing, antibacterial and anti-caries action.

In particular, it has been unexpectedly found that the dental care product according to the invention advantageously remineralizes smaller caries lesions and/or repairs microfine defects in the tooth enamel, in particular also in patients with dry mouth/saliva deficiency. Furthermore, a protective layer can be applied to the tooth by the composition according to the invention and furthermore, it has been found that the enamel exhibits advantageous solubility and resistance to acid after application of the composition according to the invention. Moreover, the present composition can dispense with antimicrobial substances (such as chlorhexidine) as excipients. Thus, excellent caries prophylaxis can be ensured without, for example, disturbing/disrupting the bacterial balance in the oral cavity and without risking unpleasant side effects.

In particular, it has been unexpectedly found that the dental care product according to the invention prevents caries and can also remineralize lesions down to deeper layers of the tooth, in particular the enamel. Furthermore, the dental care product according to the invention can apply a protective layer to the tooth and over exposed dentin and, in particular, can seal open dentinal tubules. Furthermore, it has been found that the enamel shows a significantly reduced or no longer detectable structural damage after use. In addition, the fluoride application can be completely dispensed with in the present dental care product. Thus, the above positive aspects can be ensured without, for example, disturbing/disrupting the bacterial balance in the oral cavity and without risking undesirable side effects which may occur, for example, when using oral care products containing fluoride. The present invention relates to a dental care product for mineralizing teeth, in particular enamel, to a depth of 200 μm, preferably to 100 μm (deep mineralization).

It is preferred that the at least one calcium phosphate compound is preferably in particulate form and is selected from the group consisting of monocalcium phosphate monohydrate (MCPM), monocalcium phosphate anhydrate (MCPA), dicalcium phosphate dihydrate (DCPD, brushite), dicalcium phosphate anhydrate (DCPA, monetite), octacalcium phosphate (OCP), α-tricalcium phosphate (α-TCP), β-tricalcium phosphate (β-TCP), amorphous calcium phosphate (ACP; also as CPP-ACP complex=casein phosphopeptide−amorphous calcium phosphate), amorphous calcium phosphate (ACP), calcium-deficient hydroxylapatite (CDHA), hydroxylapatite (HA or HAP), tetracalcium phosphate (TTCP) and calcium pyrophosphate, particularly preferably from hydroxylapatite and calcium pyrophosphate. The at least one calcium phosphate compound may be present in different crystal forms, including mixtures of these crystal forms, wherein rounder crystal forms are preferred over more pointed crystal forms (for example, needle-shaped crystals). Likewise, they may have different crystal and aggregate sizes, also in mixed form, and different crystallinities, likewise also in mixed form.

Hydroxylapatite (Ca₅(PO₄)₃(OH)) is also known as hydroxyapatite. It is a mineral from the mineral class of phosphates, which crystallises in a hexagonal crystal system. Furthermore, hydroxylapatite is a member of the apatite group and forms a gapless mixed series with chlorapatite and fluorapatite.

A Ca₅(PO₄)₃(OH) suitable according to the invention is described, for example, in DE 10 2016 114 189.5. The Ca₅(PO₄)₃(OH) used according to the invention is preferably produced synthetically. This means that the Ca₅(PO₄)₃(OH) used according to the invention is preferably not obtained by burning out the organic components from animal material such as bones.

The calcium phosphate compounds used according to the invention can be present both in pure form and in the form of substituted compounds as well as mixtures thereof. According to the invention, a pure form is present when the ions contained in the respective calcium phosphate compound are in each case substituted by less than 1%, preferably less than 0.5%, even more preferably less than 0.1% by one or more other ions. For example, in pure hydroxylapatite, the Ca²⁺ ions are substituted by, for example, Mg²⁺ or Zn²⁺ to less than 1%, preferably less than 0.5%, even more preferably less than 0.1%.

Further preferably, the calcium phosphate compounds do not contain any doping according to the invention, such as a zinc carbonate doping. However, doped calcium phosphate compounds can also be used.

The X₅₀ value of the volume-based particle size distribution of the calcium phosphate compounds is 1.0 nm to 100.0 μm, preferably 10 nm to 10.0 μm, more preferably 50 nm to 1 μm, particularly preferably 100 nm to 5500 nm, wherein the X₅₀ value of the volume-based particle size distribution is measured by means of laser diffraction.

For this purpose, a sample of the calcium phosphate compounds is first sonicated in an ultrasonic homogenizer with an energy output of 96 W for 9 minutes and then for another 3 minutes in a sample preparation device. The subsequent particle size distribution measurement (laser diffraction) is carried out in a particle size determination instrument at a temperature of 25° C.±0.3° C. and the corresponding values are calculated according to the Mie theory. The measuring instruments used are exclusively commercially available devices.

In a preferred embodiment according to the invention, the calcium phosphate compound is Ca₅(PO₄)₃(OH) and has a hexagonal crystal lattice in which the length of the α-axis is 0.930 to 0.950 nm, preferably 0.933 to 0.948 nm, particularly preferably 0.936 to 0.945 nm, and the length of the c-axis is 0.680 to 0.700 nm, preferably 0.682to 0.696 nm, particularly preferably 0.685 to 0.692 nm. The lengths of the α-axis and of the c-axis are determined by a Rietveld evaluation of the corresponding X-ray powder diffractograms. The X-ray powder diffractograms themselves are obtained by means of a measurement with a conventional powder diffractometer at the routine settings.

In a preferred embodiment of the invention, the at least one calcium phosphate compound is present in aggregated form. In this case, an aggregation is understood to be an agglomeration of molecules or particles to form a larger association, the aggregate. This agglomeration or aggregate is caused and held together by various forces and/or types of bonding, such as ionic bonding, Van der Waals forces, intermolecular forces, or other types of chemical bonding. The degree of aggregation and also the size of the aggregate can be determined with the aid of scanning electron microscopy.

The dental care product according to the invention comprises calcium phosphate compounds in an amount of 0.01 to 80% by weight, preferably 0.1 to 20% by weight, particularly preferably 1 to 10% by weight, and in particular 1 to 5% by weight, in relation to the total weight of the dental care product. For mouth rinses, the preferred amount of calcium phosphate compounds is 1 to 5% by weight, for toothpastes 10 to 20% by weight, in each case in relation to the total weight.

Preferably, the at least one surfactant is selected from the group consisting of sodium methyl cocoyl taurate, sodium cocoyl glycinate, sodium lauroyl sarcosinate and sodium myristoyl sarcosinate.

The dental care product according to the invention preferably contains at least one calcium compound selected from the group consisting of calcium carbonate, calcium chloride, calcium bromide, calcium nitrate, calcium acetate, calcium gluconate, calcium lactate, calcium tartrate and also hydrates and mixtures thereof, preferably calcium carbonate.

The dental care product according to the invention preferably does not contain perlite. Perlite is a cleaning body with a high relative hardness and may damage the teeth.

The dental care product according to the invention preferably does not contain any aluminum compounds. Aluminum compounds can be potentially toxic.

The dental care product according to the invention preferably does not contain diamond particles. Diamond is a cleaning body with a high relative hardness and may damage the teeth.

The dental care product according to the invention preferably does not contain cellulose fibers (microcrystalline cellulose=cleaning bodies). Cellulose fibers often lead to an unpleasant mouthfeel, and therefore user compliance is severely limited.

The dental care product according to the invention preferably does not contain sodium hydrogen carbonate. Sodium hydrogen carbonate has only a very low cleaning power.

The dental care product according to the invention preferably does not contain glucosides. Glucosides can break down glucose, which promotes the undesirable development of caries.

The oral care product according to the invention does not contain abrasive polymer particles/“microbeads” (for example polyethylene beads). Such polymer particles may have harmful effects on the environment.

The dental care product according to the invention preferably does not contain any sulfates, such as sodium lauryl sulfate, or sulfonates.

The dental care product according to the invention is used for the remineralization of teeth up to a depth of 200 μm, preferably up to 150 μm, in particular up to 100 μm. Such remineralization of teeth down to these depths is referred to as deep remineralization, since in this case not only the tooth surface areas down to a depth of approximately 30 μm, as described in the prior art, but also deeper areas of the tooth are remineralized.

It has been found that the dental care product according to the invention can be used in the treatment and/or prevention of numerous dental diseases.

In a preferred embodiment of the invention, the dental care product according to the invention can be used for the treatment of (dental) diseases/conditions selected from caries, dental erosion, dental abrasion/, attrition, bruxism, molar incisor hypomineralization (MIH), amelogenesis imperfecta, dentinogenesis imperfecta and fluorosis.

The term “caries” is well known to professionals. Caries is generally understood to be a destructive disease of the hard tissues of the tooth, enamel and dentin.

Dental erosion refers to damage to the tooth structure caused by acids, in other words defects in the enamel and/or dentin caused by dental erosion, which, if treated too late, can lead to irreversible damage.

(Tooth) abrasion is the loss of hard tooth substance through friction. Attrition is a sub-form of abrasion, specifically the loss of hard tooth substance by reflexive contact with the teeth.

Bruxism is the unconscious grinding or clenching of the teeth, usually at night but also during the day, which may result in wear and tear not only of the teeth but also of the periodontium and chewing muscles.

Molar incisor hypomineralization (MIH), which is also known as “chalk teeth”, is an enamel disorder, i.e. structural damage to the enamel.

Amelogenesis imperfecta is considered to be a genetically induced disease in which there is a disturbance in the formation of tooth enamel. As a result, the teeth have an increased risk of caries formation and are particularly sensitive to temperature.

Dentinogenesis imperfecta is an autosomally dominant, inherited maldevelopment/structural disorder of the tooth dentition that occurs in approximately 1 in 8000 people and results in severe abrasion of the teeth.

Tooth (fluorosis) (also: dental fluorosis) is understood to be a non-inflammatory disease (“speckled teeth”) caused by excessive fluoride intake, in particular during the ontogenetic development of the teeth. It has been found that the use according to the invention can prevent the above-mentioned (dental) diseases and/or at least significantly slow down their progression and/or achieve complete restoration of the tooth substance, in particular the hard tooth substance. In particular, it has been found that the use according to the invention, or after its application, results in a significantly reduced or no longer detectable structural damage of the tooth enamel.

In a preferred embodiment, the dental care product according to the invention can be used to treat a code 3 or code 4 caries, preferably a code 3 caries, determined according to the International Caries Detection and Assessment System (ICDAS).

According to the International Caries Detection and Assessment System (ICDAS), caries is classified into different codes (levels), wherein the higher the code, the more severe the decay on the tooth and consequently its impact on that tooth.

In the case of a code 0 caries, no signs of caries are visible after drying in the air stream for about 5 seconds.

In the case of a code 1 caries, the first visual changes in the enamel surface are visible after the tooth has dried. The changes may be opacities or whitish or brownish discolorations.

In the case of a code 2 caries, clear visual changes in the enamel surface are already present on the moist tooth. These changes may be opacities in the sense of a white spot lesion and/or brownish carious discoloration in the fissures/grooves and must still be visible on the dried tooth.

In the case of a code 3 caries, demineralization or loss of enamel structure is present without visible changes in the dentin. The opacities and/or brownish or black carious changes extend beyond the border of the fissures/pits and are visible even after the tooth has dried. If necessary, a WHO probe can be carefully passed over the enamel defect to palpate the discontinuity of the enamel surface.

In the case of a code 4 caries, there is shadowing in the dentin, with or without enamel collapse. The shadowing may be greyish, bluish or brownish.

In the case of a code 5 caries, clear cavity formation with visible dentin is visible. Enamel loss is clearly visible on the dried tooth. If necessary, the WHO probe can be used to feel the exposed dentin.

In the case of a code 6 caries, there is extensive cavity formation, wherein the dentin is clearly visible across the width and depth of the tooth. At least half of the enamel surface is cariously destroyed. The pulp may be affected.

In a preferred embodiment, the dental care product according to the invention can be used for both cosmetic and medical purposes. This means, for example, that it can be used not only for the treatment of the above-mentioned (dental) diseases, but also for cosmetic purposes, such as beautifying the appearance of the teeth.

In a preferred embodiment, the dental care product according to the invention is used on persons of all ages, including children, and, in the case of children, preferably on children aged 6 months to 14 years, in particular on children aged 10 months to 12years.

In a preferred embodiment, the Ca₅(PO₄)₃(OH) contained in the dental care product according to the invention is the only apatite component of the dental care product.

In a preferred embodiment, the dental care product according to the invention comprises 0.01 to 80% by weight, preferably 0.2 to 40% by weight, more preferably 0.5 to 30% by weight, in particular 1.0 to 20% by weight of calcium phosphate compounds. In a preferred embodiment of the invention, the dental care product according to the invention can comprise 1% by weight, 2% by weight, 3% by weight, 4% by weight, 5% by weight, 6% by weight, 7% by weight, 8% by weight, 9% by weight, 10% by weight, 11% by weight, 12% by weight, 13% by weight, 14% by weight, 15% by weight, 20% by weight or 25% by weight of calcium phosphate compounds.

In a preferred embodiment, the dental care product according to the invention comprises one or more calcium salts which have a solubility of at least 10 mg/l H₂O at 20° C. The solubility is determined by methods known to a person skilled in the art or can be deduced from the relevant technical literature.

In a preferred embodiment, the dental care product according to the invention may contain one or more pharmaceutical or cosmetic ingredients. These pharmaceutical or cosmetic ingredients are described, for example, in Toothpastes, Monographs in Oral Science, Vol. 23, 1st edition, 2013.

Preferably, the one or more pharmaceutical or cosmetic ingredients comprise xylitol, antimicrobial substances, pH regulators, abrasives, flavorings and humectants, in particular xylitol, pH regulators, abrasives and flavorings.

Xylitol can minimize the number of caries bacteria and inhibit their growth. Furthermore, xylitol can stimulate the flow of saliva. The increased amount of saliva results in an increased amount of phosphate. This phosphate can react with the calcium (ions) from the dental care product according to the invention to form hydroxylapatite. The dental care product according to the invention may contain xylitol in an amount of 0.5 to 15% by weight, preferably 1 to 10% by weight, in particular about 7.0% by weight, in relation to the total weight of the dental care product. In addition to xylitol, the dental care product according to the invention may contain other sugar alcohols such as sorbitol.

Antimicrobial substances are substances that can kill microorganisms, such as bacteria, or greatly reduce their proliferation. In addition to antimicrobial substances with a non-specific defense against bacteria and fungi, there are also substances that are only effective, for example, against specific bacteria. The use of antimicrobial substances can also combat bad breath, for example. Preferably, antimicrobial substances can be contained in the dental care product according to the invention in an amount of 0.01 to 2.0% by weight, preferably 0.05 to 1.0% by weight. Examples of antimicrobial substances used in oral care are zinc compounds, such as zinc chloride and zinc citrate, as well as essential oils and surfactants.

In a particularly preferred embodiment, the dental care product according to the invention does not contain chlorhexidine.

In a particularly preferred embodiment, the dental care product according to the invention does not contain triclosan.

pH regulators are substances that can establish a certain pH value range, preferably a range of pH 6.5 to 7.5. If the product is too acidic, there would be a risk of demineralization of the hard tooth structure (erosion). Examples of pH regulators are sodium hydroxide (NaOH) or phosphoric acid (H₃PO₄), which can be used according to the desired pH value. Sodium hydroxide can be added to raise a pH value that is too low, while phosphoric acid can be added if the pH value is too high. pH regulators can be present in up to 5% by weight in relation to the total weight of the dental care product according to the invention.

In a preferred embodiment, the dental care product according to the invention contains one or more flavorings, which can give it the desired flavor. This one flavoring or these several flavorings can be natural, nature-identical, synthetic flavorings and/or mixtures thereof. Examples of flavorings are limonene, geraniol, citronellol and eugenol. In addition, flavorings can be saliva stimulants, wherein the moisture of the saliva can have a positive influence on the remineralization of the tooth. An example of a salivα-stimulating flavoring is pellitorin, especially trans-pellitorin.

Flavorings may preferably be present in the dental care product according to the invention in an amount of 0 up to 5% by weight, preferably 0.1 to 3% by weight, in relation to the total weight of the dental care product according to the invention.

Humectants are additives that prevent the dental care product according to the invention from drying out by binding water added during the production process (i.e. preventing evaporation) or by attracting moisture from the air during storage. Examples of humectants include glycerol, propane-1,2-diol, hexane-1,2-diol, egg yolk, aloe vera gel, honey, molasses, in particular glycerol and hexane-1,2-diol. Humectants may preferably be present in the dental care product according to the invention in an amount of 0 to 25% by weight, preferably 0.1 to 20% by weight, in relation to the total weight of the dental care product according to the invention.

In a preferred embodiment, the dental care product according to the invention contains
0.01 to 80% by weight, preferably 0.2 to 40% by weight, more preferably 0.5 to 30% by weight, in particular 1.01 to 20% by weight of calcium phosphate compounds, preferably hydroxylapatite,
0.3 to 3% by weight of surfactants, preferably about 1.0% by weight of sodium cocoyl glycinate or sodium methyl cocoyl taurate,
0.5 to 15% by weight, preferably 1 to 10% by weight, in particular about 7.0% by weight of xylitol,
0 to 2.0% by weight, preferably 0.01 to 2.0% by weight, more preferably 0.05 to 1.0% by weight of antimicrobial substance, in particular zinc chloride,
0 to 5% by weight, preferably 0.3 to 2.0% by weight of pH regulator, in particular phosphoric acid,
0 to 5% by weight, preferably 0.1 to 3% by weight of flavoring,
0 to 25% by weight, preferably 0.1 to 20% by weight of humectant, in particular glycerol and/or hexane-1,2-diol,
   wherein the values in % by weight are in relation to the total weight of the dental care product. The remainder is distilled water, if applicable.

The pH value is in a neutral range of pH 6.5 to 7.5.

The invention is explained below by means of examples.

Exemplary embodiments for the composition of dental care products according to the invention in the form of toothpastes (in % by weight):

| | Exemplary | Exemplary | Exemplary | Exemplary |
|---|---|---|---|---|
| | embodiment 1 | embodiment 2 | embodiment 3 | embodiment 4 |
| Demineralized water | 44.2 | 56.5 | 55.2 | 50.7 |
| Glycerol | 10.0 | 5.0 | 10.0 | 14.0 |
| Sorbitol | 4.0 | 3.0 | 10.0 | 6.0 |
| Hydroxylapatite | 30.0 | 20.0 | 10.0 | 5.0 |
| Xylitol | | 2.0 | 8.0 | 10.0 |
| Cellulose gum | 7.0 | 1.0 | 1.0 | 3.0 |
| Hydroxyethyl cellulose | | 0.2 | 1.0 | 0.5 |
| Carboxymethyl cellulose | 0.1 | 0.2 | 1.0 | 0.5 |
| Sodium methyl cocoyl taurate | 1.0 | 2.0 | 0.5 | 2.0 |
| Sodium sulfate | | | 0.5 | |
| 1,2-hexanediol | | | 0.2 | |
| Caprylyl glycol | | | 0.5 | |
| Flavoring | 0.5 | | 0.3 | 1.5 |
| Sodium cocoyl glycinate | | | 0.2 | |
| Sodium chloride | | 0.7 | | 0.3 |
| Benzyl alcohol | | 0.5 | | |
| Phenoxyethanol | 0.3 | 0.4 | 0.1 | 0.3 |
| Potassium sorbate | 0.2 | | | |
| Sodium benzoate | | 1.0 | | 1.0 |
| Allantoin | | | 0.5 | 1.0 |
| Sodium myristoyl sarcosinate | 1.0 | 1.5 | | 2.0 |
| Sodium saccharin | 0.5 | 0.5 | 0.5 | 0.5 |
| Tocopheryl acetate | | 0.1 | | 1.0 |
| Zinc chloride | 0.1 | 0.1 | | 0.5 |
| Carrageenan | 0.1 | 3.0 | | 0.5 |
| Xanthan gum | | 0.2 | 0.3 | 0.3 |
| Total | 100.0 | 100.0 | 100.0 | 100.0 |

### STUDY

A study was carried out on the properties of a toothpaste according to the invention.

### Example 1

A toothpaste not according to the invention was prepared which contained the following ingredients:
demineralized water 55.30% by weight,
hydrogenated starch hydrolysate 21.60% by weight,
hydrated silica 2.00% by weight (Grace, SM 850 C)
hydroxylapatite 10.00% by weight,
xylitol 7.00% by weight,
sodium carboxymethyl cellulose 0.80% by weight,
hexane-1,2-diol 0.40% by weight,
flavoring 0.40% by weight.

The toothpaste is a light beige, homogeneous, creamy paste with an average pH value of 7.2.

### FIGURE LIST

FIG. 1 shows a flow chart with the stepwise methodology. This is a crossover study, so for the 30 subjects who fully completed the study, the two interventions were performed in a crossover design as Phase I & II.

FIG. 2 shows representative microradiographic images of healthy tooth tissue before (A) and after (B) inter-oral exposure to demineralization while the subject is using a children's toothpaste (Karex) containing 10% hydroxylapatite in the form of microclusters.

FIG. 3 shows representative microradiographic images of healthy tooth tissue before (A) and after (B) inter-oral exposure to demineralization while the subject is using a children's toothpaste (Elmex) containing 500 ppm fluoride as amine fluoride.

FIG. 4 shows representative microradiographic images of subsurface enamel lesions (initial caries lesions) before (A) and after (B) in situ remineralization by treatment with a children's toothpaste (Karex) containing 10% hydroxylapatite in the form of microclusters. FIG. 5 shows representative microradiographic images of subsurface enamel lesions (initial caries lesions) before (A) and after (B) in situ remineralization by treatment with a children's toothpaste (Elmex) containing 500 ppm fluoride as amine fluoride.

### NATURE AND MANNER OF THE STUDY

In a randomized, monocentric, in situ controlled, crossover, double-blind study, two children's toothpaste compositions containing either 10% hydroxylapatite in the form of microclusters or 500 ppm fluoride provided as amine fluoride (AMF) were compared in respect of their ability to induce remineralization and prevent the development of initial caries lesions.

### Test Subject Selection

The study included 32 people of different ethnicity aged 18 to 60 years, who were not taking antibiotics or medications that affect salivary flow and had at least 20 natural, non-crowned teeth, and who had a history of caries but no clinically active caries at the start of the study. In addition, the subjects had no dental and/or gum disease, were not pregnant or breastfeeding, and did not smoke tobacco products.

Creation of artificial initial caries and production of the in situ device 32 freshly extracted human deciduous teeth without caries, cracks or enamel defects were selected and cleaned. Four tooth blocks were made from buccal and lingual surfaces of each of the selected teeth, wherein each of the blocks was approximately 2 mm long, 2 mm wide and 1.5 mm deep. Two blocks were retained as healthy blocks for the assessment of demineralization prevention, while initial caries was artificially induced in the two blocks determined for the assessment of remineralization. All sides of each block were coated in two layers of acid-resistant nail varnish except for the buccal and lingual surfaces, respectively, where an initial caries lesion (demineralization) was produced by exposing the exposed surface to an acidified gel system (0.1 M lactic acid, 0.1 M sodium hydroxide, 60% w/v hydroxyethylcellulose, pH 4.5) for seven days. The nail varnish was then carefully removed with acetone. A tooth potion approximately 150 μm thick was cut from each tooth block to measure the baseline mineral loss (Δz₁) and lesion depth (LD1) of each induced initial caries lesion and to select lesions suitable for remineralization assessment. The portions were prepared for transverse microradiography as follows. Both sides of the portions were polished using a lapping film in an Allied High Tech MultiPrep™ precision polishing machine to create plane-parallel surfaces and reduce the thickness of the portions to 100 μm. The portions were then microradiographed on a type 1A high-resolution glass X-ray plate from Microchrome Technology, CA, USA using a Philips X-ray generator with the appropriate settings. The plates were exposed to radiation for 10 minutes at an anode voltage of 20 kV and a tube current of 10 mA and then further processed, wherein the further processing consisted of five minutes of development in a Kodak HR developer, fifteen minutes of fixation with a fixing agent (Kodak Rapid-fixer) and thirty minutes of washing. After drying, the microradiographs were examined under an optical microscope (Leica DMR) connected to a PC via a camera (Sony; model XC-75CE CCTV). Using image analysis software (TMR2006 version 3.0.0.11; Inspector Research Systems, Amsterdam), the magnified image of the microradiographs were analyzed under standard conditions of light intensity and magnification together with the image of a step wedge described in the literature. Then, the images were used only to select the lesions suitable for the comparative test. Only the samples that showed caries-like surface lesions, which showed a reasonably uniform width along their length, were selected for the remineralization process. Their blocks were used for the in situ application device.

As mentioned above, the four blocks from each tooth were distributed as follows: two blocks with lesions for remineralization assessment and two blocks for demineralization inhibition assessment. These blocks were used to fabricate the in situ device as follows. Each block was covered with a polyester fiber (Bard Peripheral Vascular, Inc. Tempe, AZ, USA) and mounted in an orthodontically adapted fixture. The device consisted of an orthodontic molar pad with retaining mesh lining (American Orthodontics Corp., Sheboygan, USA) with a ring of 0.7 mm orthodontic wire bent so that the ring tightly enclosed each test block. Each device was sterilised with gamma radiation before being given to the test subject.

### Conducting the Study

The study was conducted in two different treatment phases in which the test subjects were subjected to one of the following two treatments in a randomized, crossover comparison: (A) toothpaste containing 10% hydroxylapatite in the form of microclusters (Kinder Karex, Dr Kurt Wolff GmbH & Co. KG, Bielefeld, Germany and (B) toothpaste containing 500 ppm fluoride provided as amine fluoride (AMF) (Elmex Kinderzahnpasta, GABA GmbH, Hamburg, Germany). A one-week rinsing phase was followed by a four-week treatment phase consisting of two two-week phases, during which each test subject applied their assigned treatment under the following conditions: the first two-week phase for the test subjects wearing the in situ device with healthy enamel block, and the second two-week phase for the test subjects wearing the in situ device with enamel block with lesions.

The test subjects who met the inclusion criteria were given a specially prepared rinsing toothpaste for the one-week rinsing phase for two-minute application twice a day (morning and evening), which contained neither hydroxylapatite nor fluoride.

After the rinsing phase, the test subjects were assigned either to the group using hydroxylapatite or to the group using amine fluoride by the coordinator, who assigned randomized numbers generated by a computer program. However, to ensure that both the individuals conducting the trial and the test subjects were blind to the product, all toothpaste tubes were packaged and coded identically by the manufacturing/packaging company. After randomization, the four block-carrying in situ devices, which came from one tooth, were assigned to one test subject. Thereafter, the first of the four assigned devices was fixed to the buccal surface of the selected lower molar by a qualified dentist in accordance with standard orthodontic practice. To secure the device, the buccal surface of the selected tooth was carefully etched for 30seconds, washed with water, dried for 30 seconds and isolated using cotton wool swabs. Transbond™ XT light cure adhesive paste (3M Unitek, Monrovia, Ca, USA) was applied to the underside of the device and carefully placed. The excess material exiting at the sides was used to cover the sides, and the adhesive paste was cured by applying an Ortholux XT (3M Unitek, Monrovia, CA, USA) for 20 seconds. After attaching the device, each test subject was given their test toothpaste and a special soft toothbrush. The subjects were instructed to continue with their routine of brushing their teeth twice a day for two minutes using only 10 millilitres of water for rinsing. In addition, specific instructions were given on how to spread the toothpaste, and the test subjects were asked not to brush the device directly, and not to eat or drink anything for at least 30 minutes after cleaning, as well as to refrain from using other oral hygiene products (such as mouthwash, chewing gum, etc.). As a control, each subject was provided with a diary to record the time of each brushing phase and the weight of the toothpaste tubes was determined. After two weeks, the device was removed from the test subjects without having used the test toothpaste that morning and were sent to the laboratory for analysis. The device was attached for the second two-week treatment phase. After completion of the second two-week treatment phase, the second device was removed from the test subjects and the subjects were given a rinse toothpaste and soft-bristled toothbrush so that they could undergo a seven-day rinsing phase without the device in preparation for phase 2 of the study. After completion of this rinsing phase, the phase 1 procedure was repeated to complete the second two-week treatment, so that each subject had gone through both arms of the study.

### Post-Study Procedure and Study Exit

After inter-oral exposure, a portion approximately 150 μm thick was cut from each healthy and each lesion-containing tooth block and processed for microradiography as previously described for the baseline control portions. Although the lesion-containing control portions were microradiographed to select appropriate lesions, they were microradiographed again along with post-test portions to quantify Δz and LD of the lesions, as for the baseline portions. This step allowed control and test portions from the same block to be microradiographed and analyzed under the same conditions. For the lesion-containing portions, this process gave the pre-test mineral loss (Δz1) and lesion depth (LD1), the post-test mineral loss (Δz2) and lesion depth (LD2), and the pre-and post-test microradiograms for the lesions. For the healthy portions, this process gave the post-test mineral loss (Δz) and lesion depth (LD) and the pre-and post-test microradiograms. Using the microradiograms, the pattern and extent of remineralization in each lesion produced by the treatment by each treatment arm was examined by comparing the pre-and post-test images. For each test subject, the post-treatment mineral loss was subtracted from the pre-treatment mineral loss, and then standardised among the test subjects by dividing this difference by the pre-treatment mineral loss to obtain the remineralization in the form of a percentage. The depth of lesions before and after treatment was handled in the same way to obtain the reduction in lesion depth in %. The two toothpastes used were compared using these values.

### Analysis and Calculation of Sample Size

Sample size calculations were performed using nQuery Advisor software (Statistical Solutions, Cork, Ireland). Based on previous studies in which the mean remineralization in percentage was 30.3 with a standard deviation of 16.3 and on the assumption that each of the two toothpaste products promoted remineralization and a reduction in lesion depth significantly greater than zero, an effective sample size of 30 test subjects has a power of 0.95 with a one-tailed 0.05 significance level. Hereby, using a two-tailed t-test of two independent means, a difference between a This text has been adapted by the German Patent and Trade Mark Office (DPMA) from original sources. It does not contain any drawings. The presentation of tables and formulas may be unsatisfactory. Hypothetical means of zero and a sample mean of remineralization equal to or greater than 10% are determined. However, to provide a 5% failure, 32 subjects were included.

### Statistical Analysis

Three endpoints were determined in each case to determine mineral loss and lesion depth (1). The mean amount of remineralization and the mean amount of lesion depth reduction were determined for the hydroxylapatite-containing toothpaste (Karex) as the respective percentage of pre-treatment mineral loss and pre-treatment lesion depth. These percentages were compared to a value of 0, which is the expected value of a toothpaste without any effect. The statistical test used was a one-tailed t-test of a group mean. (2) In the same way, the mean amount of remineralization and the mean amount of lesion depth reduction were determined for the amine fluoride-containing toothpaste (Elmex) and were also compared to 0. (3) The primary endpoint was taken using the two-tailed t-test of two independent means to compare the means of the hydroxylapatite-containing toothpaste (Karex) with the means of the amine fluoride-containing toothpaste (Elmex). Equivalence was established when the difference between the two toothpaste compositions was considered clinically irrelevant for each measurement method and with Δ≤20%, wherein the statistical package R, version 3.5.0 was used for the analysis.

Results

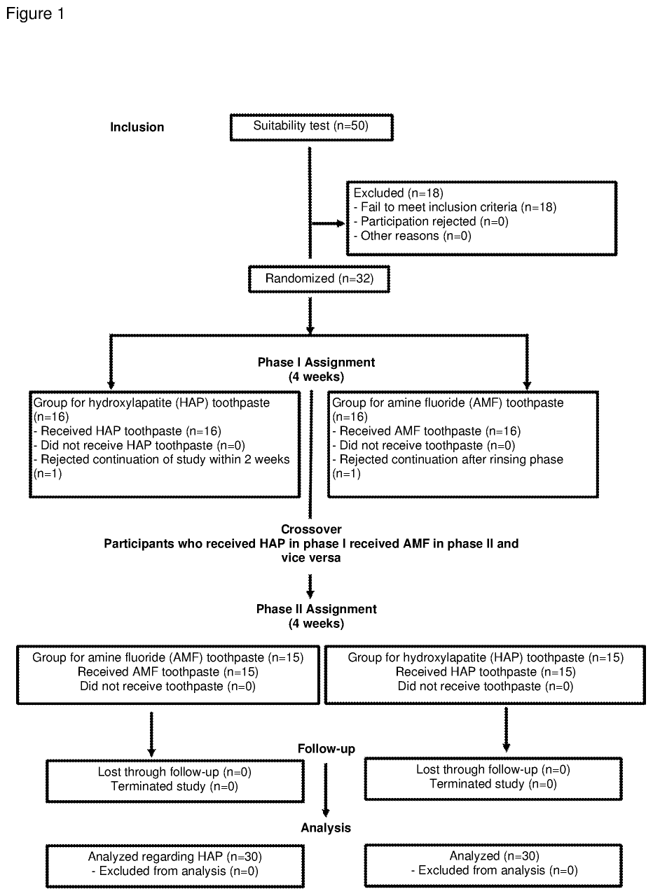

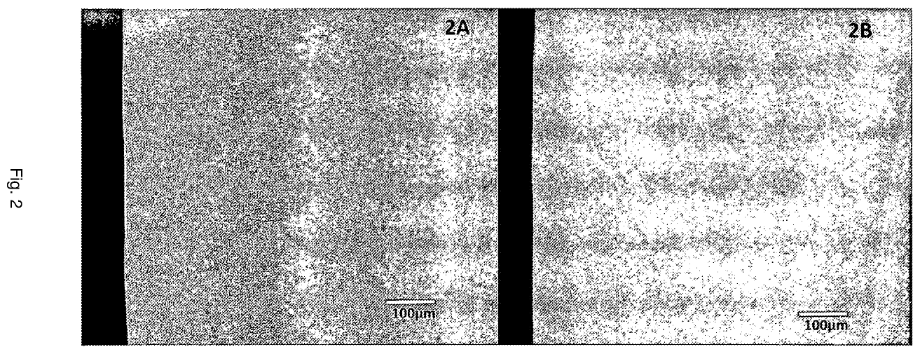

As can also be seen from FIG. 1, two test subjects dropped out of the study: one during/after the rinsing phase and one during the first two-week treatment phase while wearing the device. The study was completed by 30 test subjects (19 female and 11 male) of different ethnicity with an average age of 39.5 years.

The mean rate of remineralization and lesion depth reduction is shown in Table 1 below.

TABLE 1
Mean rates of remineralization and lesion
depth reduction in % for each toothpaste

| | 10% HAP | 500 ppm amine | p-value, 2 |
|---|---|---|---|
| Measurement | (Karex) | fluoride (Elmex) | mean values |
| Remineralization (%) | 55.8 | 56.9 | 0.81 |
| | (S.D. 13.8) | (S.D. 14.9 | |
| p-value, one group | <0.0001 | <0.0001 | |
| Lesion depth | 27.1 | 28.4 | 0.68 |
| reduction (%) | (S.D. 10.6 | (S.D. 9.8 | |
| p-value, one group | <0.0001 | <0.0001 | |

As can be seen from the table above, each of the toothpastes shows a remineralization of more than 50% and a lesion depth reduction of more than 25%.

For both toothpastes, the mean remineralization and mean lesion depth reduction were statistically significantly greater than 0. When compared with each other, there was no statistically significant difference in remineralization (p=0.81) or lesion depth reduction (p=0.68). The 95% confidence interval of the difference between HAP (Karex) and amine fluoride (Elmex) for mineralization was −8.8% to 6.5%, and the 95% confidence interval of the difference between HAP (Karex) and amine fluoride (Elmex) for lesion depth reduction was −6.8% to 4.1%.

Consequently, this study confirms that a HAP-containing toothpaste is not inferior in effectiveness to a fluoride-containing toothpaste.

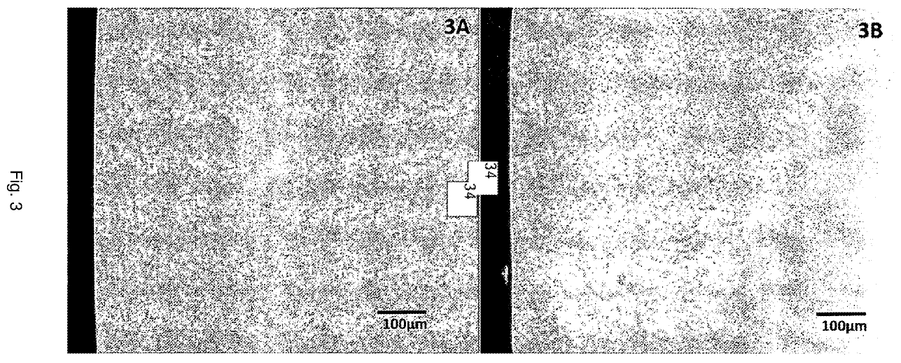

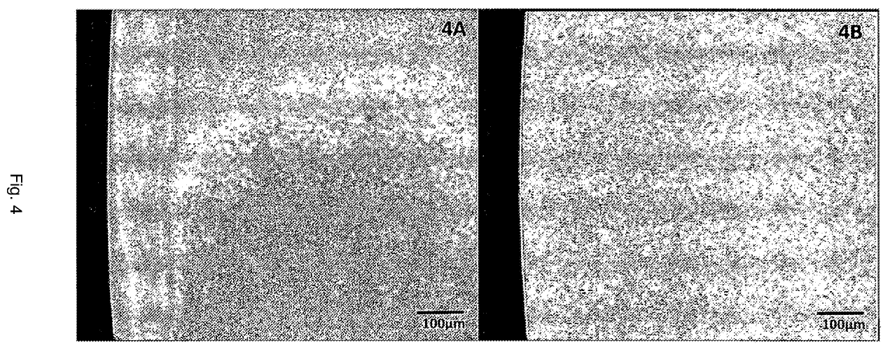

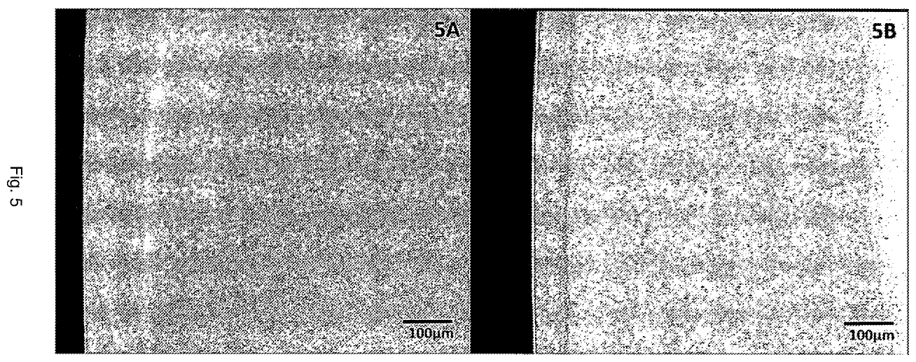

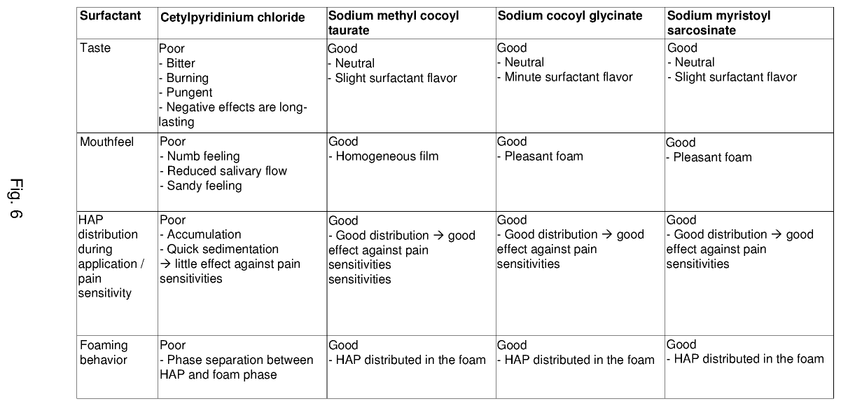

In the analysis of the healthy tooth blocks examined in respect of the ability of the two toothpastes to prevent demineralization of healthy tooth surfaces, there was no evidence of demineralization in any of the tooth blocks after intrα-oral exposure to either toothpaste, as shown in FIG. 2A & 2B and 3A & 3B. A differential comparison of the microradiograms of the lesion-containing samples exposed to the treatment of the toothpastes containing HAP (FIG. 4A & FIG. 4B) and amine fluoride (FIG. 5A & FIG. 5B) with the corresponding control microradiograms revealed the following. While the toothpaste containing HAP caused more homogeneous remineralization across the entire thickness of the subsurface lesion (FIG. 4B), the remineralization caused by the toothpaste containing amine fluoride was denser in the outer half (surface area), so that two areas of different density are clearly visible in FIG. 5B. Overall, no incidents of adverse effects were reported or clinically observed by the test subjects.

In conclusion, a HAP-containing toothpaste was found to be equivalent to a fluoride-containing toothpaste in terms of remineralization and lesion depth reduction, but without the aforementioned negative side effects that may accompany the use of a fluoride-containing toothpaste.

Moreover, in contrast to a fluoride-containing toothpaste, which prevents demineralization and causes remineralization in the surface area up to about 30 μm, a HAP-containing toothpaste can be used to prevent demineralization and also to remineralize areas lying below the surface, such as in the case shown of about 100 μm. This can improve the resistance of the teeth to dental diseases.

Subsequently, the abrasiveness of the toothpaste according to Example 1 was compared with a corresponding toothpaste in which the ingredient hydrated silica (abrasive) was replaced by glycerol (non-abrasive) and which otherwise had the same composition as that of Example 1:

### Example 2

A toothpaste according to the invention was produced which contained the following ingredients:
demineralized water 55.30% by weight,
hydrogenated starch hydrolysate 21.60% by weight,
glycerol 2.00% by weight
hydroxylapatite 10.00% by weight,
xylitol 7.00% by weight,
sodium cocoyl glucinate 0.80% by weight,
hexane-1,2-diol 0.40% by weight,
flavoring 0.40% by weight.

The toothpaste is a light beige, homogeneous, creamy paste with an average pH value of 7.2.

With the toothpastes according to Example 1 and according to Example 2, the abrasion behavior on polymethyl methacrylate plates was carried out as described in “The use of a profilometer for both quantitative and qualitative measurements of toothpaste abrasivity”, Int. J. Dent. Hygiene 8, 2010, 237-243.

In the course of this study, it was surprisingly found that the values for the removed volume for the toothpaste according to Example 2 were within the standard deviation of those of Example 1, and thus the abrasiveness of both toothpastes is comparable.

With the toothpastes according to Example 1 and according to Example 2, both the dentin abrasiveness and enamel abrasiveness were performed as described in “Dentistry-Dentifrices-Requirements, test methods and marking”, EN ISO 11609.

In the course of this study, it was surprisingly found that both the RDA (radioactive dentin abrasion) and REA (radioactive enamel abrasion) values of both toothpastes according to Example 2 were within the standard deviation of those of Example 1 and thus the abrasiveness of both toothpastes on dentin and enamel is comparable.

A comparative study was carried out with the toothpastes according to Example 1 and according to Example 2 as described in “In vitro removal of stain with dental care products”, J. Dent. Res. 61, 1982, 1236-1239.

In the course of this study, it was surprisingly found that the values for the cleaning performance (pellicle cleaning ratio; PCR) of the toothpaste according to Example 2 were within the standard deviation of those of Example 1 and thus the cleaning performance of both toothpastes is comparable.

### Example 3

Four toothpastes were produced with the following formulations:

### Formulation 1:

demineralized water 55.30% by weight,
hydrogenated starch hydrolysate 21.60% by weight,
glycerol 2.00% by weight
hydroxylapatite 10.00% by weight,
xylitol 7.00% by weight,
cetylpyridinium chloride 0.80% by weight,
hexane-1,2-diol 0.40% by weight,
flavoring 0.40% by weight.

The toothpaste is a light beige, homogeneous, creamy paste with an average pH value of 7.2.

### Formulation 2:

demineralized water 55.30% by weight,
hydrogenated starch hydrolysate 21.60% by weight,
glycerol 2.00% by weight
hydroxylapatite 10.00% by weight,
xylitol 7.00% by weight,
sodium methyl cocoyl taurate 0.80% by weight,
hexane-1,2-diol 0.40% by weight,
flavoring 0.40% by weight.

The toothpaste is a light beige, homogeneous, creamy paste with an average pH value of 7.2.

### Formulation 3:

demineralized water 55.30% by weight,
hydrogenated starch hydrolysate 21.60% by weight,
glycerol 2.00% by weight
hydroxylapatite 10.00% by weight,
xylitol 7.00% by weight,
sodium methyl glycinate 0.80% by weight,
hexane-1,2-diol 0.40% by weight,
flavoring 0.40% by weight.

The toothpaste is a light beige, homogeneous, creamy paste with an average pH value of 7.2.

### Formulation 4:

demineralized water 55.30% by weight,
hydrogenated starch hydrolysate 21.60% by weight,
glycerol 2.00% by weight
hydroxylapatite 10.00% by weight,
xylitol 7.00% by weight,
sodium myristoyl sarcosinate 0.80% by weight,
hexane-1,2-diol 0.40% by weight,
flavoring 0.40% by weight.

The toothpaste is a light beige, homogeneous, creamy paste with an average pH value of 7.2.

The study included 100 people of different ethnicity aged 18 to 60 years, who were not taking antibiotics or medications that affect salivary flow and had at least 20 natural, non-crowned teeth, and who had a history of caries but no clinically active caries at the start of the study. In addition, the subjects had no dental and/or gum disease, were not pregnant or breastfeeding, and did not smoke tobacco products.

25 people each used one of the toothpastes according to formulations 1 to 4 at least twice a day for one month. They then evaluated the taste, the mouthfeel, the hydroxylapatite distribution during application as well as the effect with regard to pain sensitivity and foaming behavior. The Table shown in FIG. 6 was drawn up on the basis of the evaluation results. It shows that taurates, glycinates and sarcosinates achieve significantly better results than cetylpyridinium chloride in terms of taste, mouthfeel, pain sensitivity and foaming behavior.

1. A dental care product, in particular toothpaste, containing at least one calcium phosphate compound, wherein the dental care product contains 0.01 to 40% by weight of calcium phosphate compound(s), the dental care product is free from fluoride compounds and free from silicon compounds and contains at least one surfactant selected from the group consisting of taurates, glycinates and sarcosinates.

2. The dental care product according to claim 1, wherein the at least one calcium phosphate compound is preferably in particulate form and is selected from the group consisting of monocalcium phosphate monohydrate (MCPM), monocalcium phosphate anhydrate (MCPA), dicalcium phosphate dihydrate (DCPD, brushite), dicalcium phosphate anhydrate (DCPA, monetite), octacalcium phosphate (OCP), α-tricalcium phosphate (α-TCP), β-tricalcium phosphate (β-TCP), amorphous calcium phosphate (ACP; also as CPP-ACP complex=casein phosphopeptide−amorphous calcium phosphate), amorphous calcium phosphate (ACP), calcium-deficient hydroxylapatite (CDHA), hydroxylapatite (HA or HAP), tetracalcium phosphate (TTCP) and calcium pyrophosphate, particularly preferably from hydroxylapatite and calcium pyrophosphate.

3. The dental care product according to claim 1, t wherein the dental care product comprises calcium phosphate compounds in an amount of 0.01 to 30% by weight, preferably 0.1 to 25% by weight, particularly preferably 1 to 20% by weight, and in particular 1 to 5% by weight, in relation to the total weight of the dental care product.

4. The dental care product according to claim 1, wherein the at least one surfactant is selected from the group consisting of sodium methyl cocoyl taurate, sodium cocoyl glycinate, sodium lauroyl sarcosinate and sodium myristoyl sarcosinate.

5. The dental care product according to claim 1, wherein the dental care product contains at least one calcium compound selected from the group consisting of calcium carbonate, calcium chloride, calcium bromide, calcium nitrate, calcium acetate, calcium gluconate, calcium lactate, calcium tartrate and also hydrates and mixtures thereof, preferably calcium carbonate.

6. The dental care product according to claim 1, wherein the dental care product does not contain perlite.

7. The dental care product according to claim 1, wherein the dental care product does not contain aluminum compounds.

8. The dental care product according to claim 1, wherein the dental care product does not contain diamond particles.

9. The dental care product according to claim 1, wherein the dental care product does not contain cellulose fibers (microcrystalline cellulose).

10. The dental care product according to claim 1, wherein the dental care product does not contain sodium hydrogen carbonate.

11. The dental care product according to claim 1, wherein the dental care product does not contain glucosides.

12. The dental care product according to claim 1, wherein the dental care product does not contain abrasive polymer particles.

13. The dental care product according to claim 1, wherein the dental care product does not contain any sulfates, in particular no sodium lauryl sulfate, or sulfonates.
